(19) United States
(12) Patent Application Publication
(10) Pub. No.: US 2021/0100832 A1
(43) Pub. Date: Apr. 8, 2021

(54) INJECTABLE COMPOSITION FOR THE TREATMENT OF MUSCULOSKELETAL DISORDERS AND METHODS OF USE THEREOF

(71) Applicant: Patchor Ltd., Ness-Ziona (IL)

(72) Inventors: Inessa Solomonik, Ganey Tikva (IL); Shachar Patchornik, Ness-Ziona (IL); Dror Robinson, Kfar Shmuel (IL)

(73) Assignee: Patchor Ltd., Ness-Ziona (IL)

(21) Appl. No.: 17/060,101

(22) Filed: Oct. 1, 2020

Related U.S. Application Data
(60) Provisional application No. 62/909,290, filed on Oct. 2, 2019.

(51) Int. Cl. A61K 31/736; A61K 31/728; A61K 31/722; A61L 27/20; A61K 9/00; A61K 9/70; A61P 19/02

(52) U.S. Cl. CPC A61K 31/736; A61K 31/728; A61K 31/722; A61L 27/20; A61L 2430/00; A61K 9/0019; A61K 9/7023; A61P 19/02; A61K 9/0053

(57) ABSTRACT

Described herein are compositions and methods of use thereof for treatment of musculoskeletal disorders by way of administering at least one galectin inhibitor to a subject, such as galactoarabino-rhamnogalacturonan (GR) and derivatives thereof, and galactomannan (GM) and derivatives thereof. In particular embodiments the described compounds are administered in combination with hyaluronic acid or Chitosan.

### CROSS REFERENCE TO RELATED APPLICATIONS

Benefit is claimed to U.S. Provisional Patent Application No. 62/909,290, filed Oct. 2, 2019; the contents of which are incorporated by reference in their entirety.

### FIELD

The present invention relates to polysaccharides compositions, with or without one or more anti-inflammatory agents, and methods of preparation and uses thereof.

### BACKGROUND

Musculoskeletal diseases are a wide spectrum of diseases including but not limited to degenerative disc disease, arthritis, tendonitis, rotator cuff tear, bursitis. Degenerative Disc Disease (DDD) is a natural part of aging and over time all people will exhibit changes in their spinal discs consistent with a greater or lesser degree of degeneration. DDD occurs when a normally healthy intervertebral disc loses its flexibility, elasticity, and shock absorbing characteristics. For certain individuals a degenerated disc can cause severe constant chronic back and leg pain, and weakness due to compression of the nerve roots. The soft gel-like center of the disc (nucleus pulposus) starts to dry out and shrink. As the disc degenerates it loses height (disc height reduction), allowing the bones above and below to exert pressure on the nerve between them. Due to disc desiccation and loss of nucleus pulposus volume the disc height collapses. The shrunk gel becomes a virtual space that is potentially restorable.

Radiographic observations of DDD can be found in 40% of individuals younger than 30 and in more than 90% of individuals older than 50 years of age [11,12]. While the majority of these observations are part of the normal aging process, a subset of patients will present with symptomatic nerve root compression and chronic back pain ultimately requiring surgical intervention [13,14]. DDD can be treated pharmacologically with opiates, steroids, or non-steroidal anti-inflammatory drugs. Likewise, other conservative measures such as physical therapy and corticosteroid injections are frequently prescribed. However, these measures do not treat the underlying cause of the degenerative process and do not slow the natural progression of the disease. In progressively symptomatic patients not responsive to conservative measures, surgery is indicated. The type of intervention is based on the underlying pathology and symptomatology, ranging from discectomy to placement of an interbody graft for bony fusion. While controversial, reports of reherniation, pseudarthrosis, and adjacent segment disease can lead to recurrent symptoms and reoperations [15,16]. Prosthetic total disc replacement (TDR) devices are now being used in clinical practice as an alternative to fusion; however, multiple studies have shown that TDR devices also alter spine biomechanics significantly enough to lead to adjacent segment degeneration (ASD) [16,17]. Nucleoplasty is an emerging alternative that currently requires open surgery [18].

Arthritis is an umbrella term that refers to more than 100 different musculoskeletal diseases. Pain, which can vary in severity, is a common symptom in virtually all types of arthritis. Other symptoms include swelling, joint stiffness, and aching around the joint(s). Arthritic disorders can affect other organs in the body, leading to a variety of symptoms such as: inability to use the hand or to walk, as well as stiffness, fatigue, sleep disorder, muscle pains. These may further lead to muscle weakness, loss of flexibility, decreased fitness and reduced quality of life. Arthritis is the most common cause of disability in the United States. More than 20 million individuals with arthritis have severe limitations in function on a daily basis [1].

By far the most common type of arthritis is osteoarthritis (OA), a multifactorial disease, often-progressive, of joint degeneration, characterized by loss of cartilage, changes to the soft tissue and bone, as well as disturbances in biochemical homeostasis within the joint capsule. Symptoms include pain, joint instability, and joint fluid accumulation, and as of yet there is no cure [2]. Principal treatment of OA is focused on control of inflammation and pain. Complications related to the use of anti-inflammatory/analgesic injectable drugs [21] necessitates an alternative or improved product. Intra-articular injections of the poly-saccharide hyaluronic acid (HA) have been widely accepted as a viscosupplement for management of knee OA pain, but with controversial efficacy [21].

Saccharides are key energy and structural molecules. They are widely used in metabolic pathways and in modifications of lipids and proteins to glycolipids and glycoproteins.

There is a significant ongoing need for improving the treatment and control of musculoskeletal disorders.

### SUMMARY

Provided herein are compositions for use in treatment of a musculoskeletal disorder in a subject in need thereof, and related methods of treatment, which include administering a therapeutically effective amount of at least one galectin inhibitor selected from galactoarabino-rhamnogalacturonan (GR) and derivatives thereof, and galactomannan (GM) and derivatives thereof.

Additionally described herein is a composition which includes a chitosan mixture (CM), hyaluronic acid (HA), and GM, wherein the HA and CM are mixed together and the CM−HA−GM are at a ratio of about 0.4: 0.2: 1.0. Further compositions described herein include a composition that includes CM and HA at a ratio of about 0.6:0.3, and supplemented by GM and/or GR. In particular embodiments, the described compositions include any combination of CM, HA, GM, and GR.

The foregoing and other objects, features, and advantages will become more apparent from the following detailed description, which proceeds with reference to the accompanying figures.

### BRIEF DESCRIPTION OF THE DRAWINGS

FIG. 1 shows relative weight bearing ability of rats treated with CM−HA−GM and HA. Longitudinal data of relative weight bearing was calculated as RLt/RL14. Rats were divided into 2 test groups, 6 animals per group. 13 days of data was collected over a period of 6 weeks. Dosing of HA: injection to OA knee at day 0 and day 14 post-surgery. Dosing of HA−CM−GM: injection at day 14 post surgery. N=12. p=0.005.

FIG. 2 shows relative weight bearing of rats treated with HA, GR, and HA+GR (left to right at each time point). Rats were divided into 3 test groups, 7 rats per group: 1) HA, 2) GR, and 3) HA+GR. Data was collected at time points: prior to operation (day-1), before the treatments (day 12) and 20 days post treatment (day 34), to compare weight bearing ability on the operated OA leg relative to the total weight bearing on both hind legs. Mean+SEM. (* p=0.0012, ** p=0.0458)—Statistically significant of experimental groups compared to control. N=21.

FIG. 3 shows the nociceptive threshold resultant of a Von Frey filament procedure. Rats were divided into 2 test groups, 7 per group: HA and GR. Data shown on 1 day prior to treatment, day 1 post treatment, and day 19 post treatment. The results reflect a 2-talied t-test for independent samples were used. N=14. Day-1, p=0.3; day 1, p=0.02; and day 19, p=0.007.

### DETAILED DESCRIPTION

### I. Abbreviations

Non-steroidal anti-inflammatory
NSAID GAGs Glycosaminoglycans drug
bGP/βGP β-glycerolphosphate GFs Growth factors
BMC Bone marrow cells GP Glycerol-phosphate
BMP Bone morphogenic protein GM Galactomannan
C/GP Chitosan/β-glycerol-phosphate GR galactoarabino-rhamnogalacturonan
CM Chitosan mixture HA Hyaluronic acid
ASD Adjacent segment degeneration
TDR Total disc replacement OA Osteoarthritis
DDD Degenerative disc disease IM Intra muscular
PRP Platelet rich plasma

### II. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids, polypeptides, and small molecules are approximate, and are provided for description. Although methods and materials similar or equivalent to those described herein can be used in the practice or testing of this disclosure, suitable methods and materials are described below. The term “comprises” means “includes.” “Consisting essentially of” indicates a composition, method, or process that includes only those listed features as the active or essential elements, but can include non-active elements in addition. The abbreviation, “e.g.” is derived from the Latin exempli gratia, and is used herein to indicate a non-limiting example. Thus, the abbreviation “e.g.” is synonymous with the term “for example.” The term “about” includes minor deviations in amounts within +/−10% of the specified amount.

Definitions of common terms in the art can also be found in Expert Opinion on Drug Delivery Volume 7, 2010—Issue 10, “Advances in using chitosan-based nanoparticles for in vitro and in vivo drug and gene delivery”, Nicolas Duceppe & Maryam Tabrizian. Pages 1191-1207; Published online: 13 Sep 2010.

In case of conflict, the present specification, including explanations of terms, will control. In addition, all the materials, methods, and examples are illustrative and not intended to be limiting.

Administration: The introduction of a composition into a subject by a chosen route. Administration of an active compound or composition can be by any route known to one of skill in the art. Administration can be local or systemic. Examples of local administration include, but are not limited to, topical administration, transdermal administration in the form of patches or creams, subcutaneous administration, intramuscular administration, intrathecal administration, intrapericardial administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds, such as galectin inhibitors and agents into implantable devices, scaffolds or constructs, such as vascular stents or other reservoirs, which release the active agents and compounds over extended time intervals for sustained treatment effects.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, oral, topical, subcutaneous, intramuscular routes, or administration by inhalation, when such administration is directed at absorption and distribution throughout the body by the circulatory system.

Analog, derivative or mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure, and includes the “functional derivatives” described herein. A mimetic is a molecule that mimics the activity of another molecule, such as a biologically active molecule. Biologically active molecules can include chemical structures that mimic the biological activities of a compound. It is acknowledged that these terms may overlap in some circumstances. In particular embodiments of the claimed methods, analogs, derivatives, or mimetics having comparable activity to the expressly recited compounds can be used in place of the recited compounds.

Arthritis: An inflammatory disease that affects the synovial membranes of one or more joints in the body. It is the most common type of joint disease, and it is characterized by the inflammation of the joint. The disease is usually oligoarticular (affects few joints), but may be generalized. The joints commonly involved include the hips, knees, lower lumbar and cervical vertebrae, proximal and distal interphangeal joints of the fingers, first carpometacarpal joints, and first tarsometatarsal joints of the feet.

Chitosan: An organic polymer, which is a derivative of chitin which is formed from the deamination of chitin. Chitosan is known for its good biocompatibility, low toxicity and biodegradability. Its degradation products: oligo-chitosan, glucosamine and acetylglucosamine are beneficial to the body. These properties make it suitable for tissue engineering. Moreover, chitosan is known for its ability to increase the residence time of loaded macromolecules (proteins, hormones, antibiotics, DNA and so on) mainly through interactions between the positively charged amino groups on glucosamine units of chitosan and the negatively charged functional groups on the loaded molecules.

Chitosan is insoluble in physiological conditions, limiting its use in the body. Chemical modifications may overcome this limitation, but at the cost of decreased safety. CarGel (Smith & Nephew) found a unique way to keep insoluble chitosan in solution by adding beta-glycerolphosphate making it a bioscaffold with clinically proven safety and efficacy in a surgical procedure for articular cartilage repair that required mixing patient's whole blood with the chitosan bioscaffold at the site of surgery [4]. Ghazi Zadeh et al. 2017 [5] reported that implants composed of freeze-dried chitosan solubilized in autologous platelet-rich plasma (PRP), developed by the Canadian company Ortho Regenerative Technologies Inc, improved meniscus repair in sheep, further supporting chitosan's known tissue repair ability.

Chitosan-Mixture (CM) A mixture of consisting of type 1 chitosan, type 2 chitosan, and OligoChitin (at a ratio of about 1:1:1) as described in International Patent Application Publication No. WO 2009/150651 A1, the contents of which are incorporated by reference herein.

Chitosan-Hyaluronate hybrid gel: A gel developed as a self-forming thermo-responsive injectable hydrogel and was tested in an animal pain model of knee osteoarthritis. It was found that the intra-articular use of chitosan hybrid is possible, and that at least in an animal model it might delay osteoarthritis progression and improve knee function [23, 24].

Effective amount of a compound: A quantity of compound sufficient to achieve a desired effect in a subject being treated. An effective amount or “therapeutically effective amount” of a compound can be administered in a single dose, or in several doses, for example daily, during a course of treatment. However, the effective amount of the compound will be dependent on the compound applied, the subject being treated, the severity and type of the affliction, and the manner of administration of the compound.

Galactoarabino-Rhamnogalacturonan (GR/GR-MD-02): GR is a potent inhibitor of galectin-3 with proven anti-inflammatory and anti-fibrotic properties and binds to the carbohydrate-binding domain of galectins.

Galactomannan (GM/ GM-CT-01): GM is a carbohydrate polymer comprised of mannose and galactose with anti-inflammatory, anti-fibrotic agent properties. It is also a galectin-3 inhibitor. Galactomannan is isolated from seeds of Cyamopsis tetragonoloba.

Galectins: Galectins are carbohydrate-binding and β-galactosid-binding lectins. The function of galectins varies with their tissue-specific and subcellular location, and their binding to carbohydrates makes them key players in several intra- and extracellular processes where they bind to glycosylated proteins and lipids. In humans, there are 12 identified galectins, some with tissue-specific distribution. Galectins are found inside cells and in the nucleus, cytosol, and organelles, as well as extracellularly. Galectin-1, -2, -3, -4, -7, -8, -9, and -12 can all induce T-cell apoptosis and modulate inflammation.

Galectins are known to be involved in immunomodulation, neuroinflammation, apoptosis, phagocytosis and oxidative bursts [6]. One of the key players responsible for excessive inflammation, cartilage degradation and excessive bone remodeling is galectin-3, which has been reported to be highly expressed and secreted by inflamed synovium of rheumatoid arthritis and osteoarthritis patients. Furthermore, galectin-3 has been demonstrated to induce joint swelling and osteoarthritis-like lesions after intra-articular injection in laboratory animals [7].

Galectin-3 has a C-terminal carbohydrate recognition domain as well as an N-terminal tail. It can induce T-cell apoptosis. Galectin-3 can be both anti-apoptotic and pro-apoptotic. The N-terminal and C-terminal ends can both be involved in dimerization or possible oligomerisation of galectin-3, thought to be important for the function of the molecule. The binding properties of galectin-3 to their ligands are pH-dependent; this raises the possibility that pH could be a contributing factor in determining galectin function in different locations including the cellular microenvironments.

Galectin-3 can be endocytosed by macrophages. Uptake of galectin-3 in classically activated M1 macrophages is carbohydrate-independent and mediated by N-terminal end binding, whilst uptake in alternatively activated M2 macrophages, as well as non-macrophages, is carbohydrate dependent and involves the C-terminal CRD. In T-cells, galectin-3 is present at the cell surface associated with the TCR complex; it seems to inhibit uncontrolled T-cell activation and potentiates downregulation of TCR in T-cells. Galectin-3 is observed in among others fibroblasts, chondrocytes, osteoblasts, osteoclasts, keratinocytes, Schwann cells and gastric mucosa. It is also found in endothelial cells in a number of tissues, and in immune cells such as neutrophils, eosinophils, basophils, mast cells, Langerhans cells and dendritic cells [22].

Hyaluronic acid (HA): A high molecular weight, naturally occurring, biopopolysaccharide. It is found in most connective tissues. Sodium hyaluronate is a water-soluble salt form of hyaluronic acid developed to increase stability and lessen the likelihood of oxidization. HA has been found to act as a cushion and lubricant in the joints and other tissues. HA products are divided into two major types, native HA products and cross-linked HA products. Native HA products are injected 3 to 5 times per treatment course and their good safety profile has been established based on long-term clinical use. The cross-linked HA products were developed in order to reduce the number of injections and increase the intra-articular half-life of the Hyaluronate at the cost of reduced safety.

Injectable composition: A pharmaceutically acceptable fluid or semi-fluid composition comprising at least one active ingredient, for example, a protein, peptide, or antibody. The active ingredient is usually dissolved or suspended in a physiologically acceptable carrier, and the composition can additionally comprise minor amounts of one or more non-toxic auxiliary substances, such as emulsifying agents, preservatives, pH buffering agents and the like. Such injectable compositions that are useful for use with the compositions of this disclosure are conventional; appropriate formulations are well known in the art.

Linker: One or more nucleotides, amino acids, or other molecule that serve as a spacer between two molecules, such as between two nucleic acid molecules or two peptides. Such linkers could be used to link the described CM to the galectin inhibitor(s).

Pharmaceutically acceptable carriers: The active agents for use in the described methods can be, mixed with a pharmaceutically acceptable carrier. The pharmaceutically acceptable carriers useful in this disclosure are conventional. Remington's Pharmaceutical Sciences, by E. W. Martin, Mack Publishing Co., Easton, PA, 15th Edition (1975), and updates thereof, describes compositions and formulations suitable for pharmaceutical delivery of the compounds herein disclosed.

Pharmaceutical agent: A chemical compound or composition capable of inducing a desired therapeutic or prophylactic effect when properly administered to a subject or a cell. Incubating includes exposing a target to an agent for a sufficient period of time for the agent to interact with a cell. Contacting includes incubating an agent in solid or in liquid form with a cell.

Preventing or treating a disease: Preventing a disease refers to inhibiting the full development of a disease, for example inhibiting the development of myocardial infarction in a person who has coronary artery disease or inhibiting the progression or metastasis of a tumor in a subject with a neoplasm. Treatment refers to a therapeutic intervention that ameliorates a sign or symptom of a disease or pathological condition after it has begun to develop. Preventing and treating a disease can also refer to the results of interventions taken to prevent the recurrence of a disease that has been otherwise treated, such as surgery to replace a degraded spinal disk.

Rheumatoid arthritis: A chronic, systemic, inflammatory disease that affects the synovial membranes of multiple joints in the body. Because the disease is systemic, there are many extra-articular features of the disease as well. For example, neuropathy, scleritis, lymphadenopathy, pericarditis, splenomegaly, arteritis, and rheumatoid nodules are frequent components of the disease. In most cases of rheumatoid arthritis, the subject has remissions and exacerbations of the symptoms. Rheumatoid arthritis is considered an autoimmune disease that is acquired and in which genetic factors appear to play a role.

Subject: Living vertebrate organisms, a category that includes both human and non-human mammals

Therapeutically effective amount: A quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound. For example, a therapeutically effective amount of an active ingredient can be measured as the concentration (moles per liter or molar-M) of the active ingredient (such as a small molecule, peptide, protein, or antibody) in blood (in vivo) or a buffer (in vitro) that produces an effect.

Viscosupplementation: A procedure in which HA is injected into the knee joint, which is commonly used in order to alleviate the symptoms of osteoarthritis of the knee.

### III. Overview of Several Embodiments

Described herein are compositions for use in methods of treatment of a musculoskeletal disorder in a subject in need thereof, including human and non-human subjects, which includes a therapeutically effective amount of at least one galectin inhibitor selected from galactoarabino-rhamnogalacturonan (GR) and derivatives thereof, and galactomannan (GM) and derivatives thereof.

In particular embodiments the composition for use additionally includes hyaluronic acid (HA).

In some embodiments the composition for use further includes chitosan, such as in a chitosan mixture (CM), up to 1000 kD, or chitosan oligomers of 0.4-50 kD or glucosamine or N-acetylglucosamine or a derivative thereof.

In particular embodiments the composition for use includes an anti-inflammatory agent, anti-pain medication , including opiates, steroids, and/or anti-fibrotic agents.

In other embodiments the galectin inhibitor inhibits at least one galectin selected from the group consisting of galectin-1, galectin-2, galectin-3, galectin-4, galectin-7, galectin-8, galectin-9, and galectin-12, and particularly galectin-3.

In particular embodiments the composition for use is formulated for oral administration, parental administration, transdermal administration or by direct injection.

In some embodiments the composition for use is administered concurrently or in sequence with mesenchymal stem cells, adipose derived stromal cells, chondrocytes, platelet-rich plasma or whole blood.

In particular embodiments the composition for use is formulated for sustained release or extended release.

In other embodiments the musculoskeletal disorder is selected from the group consisting of arthritis, osteoarthritis, rotator cuff tear, degenerative disc disease, in particular osteoarthritis, more particularly osteoarthritis of the knee.

In some embodiments the composition consists essentially of chitosan mixture (CM), HA and GM, wherein the CM and HA are mixed together and the CM−HA−GM are at a ratio of about 0.4:0.2:1.0.

In particular embodiments the composition consists essentially of CM and HA at a ratio of about 0.6:0.3.

In other embodiments the composition for use further comprises GM, and optionally GR.

In some embodiments, the composition for use consists essentially GR and HA. In further embodiments, the composition for use consists essentially of GR.

Additionally described herein are compositions comprising HA, CM and GM, wherein the CM and HA are mixed together and the CM−HA−GM are at a ratio of about 0.4:0.2:1.0.

In some embodiments the composition includes CM and HA at a ratio of about 0.6:0.3, and GM or GR.

In other embodiments composition comprising CM and HA at a ratio of about 0.6:0.3, GM, and GR.

### IV. Galectin Inhibitors for Treatment of a Musculoskeletal Disorder

It is known in the literature that galectins play important roles in the immune and inflammatory responses through regulating the homeostasis and functions of immune cells [10], though as described herein, the precise role of galectin-3 in protecting or damaging tissue remains unclear. Despite the conflicting knowledge of the art, the combination of a chitosan mixture (CM) with HA and with GM or GR was tested for a possible beneficial effect in musculoskeletal diseases such as viscosupplementation for OA.

International Patent Publication No.—WO 2009/150651 A1, herein incorporated by reference, describes the combination of hyaluronic acid with insoluble chitosan in a homogenous composition through the use of a second type of soluble chitosan that “protects” the insoluble chitosan from precipitation enabling it to remain is solution (colloidal solution) at neutral pH. The described chitosan mixtures (CM) can be homogenously mixed with HA to form an injectable liquid forming gel scaffold inside the body.

Despite the conflicting evidence as to the role of Galectin-3 as causing or delaying osteoarthritis, the present disclosure describes treatments for musculoskeletal disorders by use of compositions including galectin inhibitors, such as Galectin-3 inhibitors, and which include GM or GR alone and in combination with HA or CM or HA−CM for treatment of OA. As disclosed herein, in studies of experimentally induced OA in rat, the HA−CM−GM combination surprisingly reduced pain and restored weight bearing capacity within 4 weeks from treatment, or testing GR alone or GR-HA, provided additional proof of the benefits compared to the gold-standard HA.

In particular embodiments the musculoskeletal disorder is arthritis, osteoarthritis, rotator cuff tear, degenerative disc disease, tendonitis, muscle or tendon strain, ligament sprain, tension neck syndrome, thoracic outlet compression, rotator cuff tendonitis, epicondylitis, radial tunnel syndrome, digital neuritis, trigger finger/thumb, De-quervain's syndrome, mechanical back syndrome, ruptured or herniated disc, and repetitive motion injury rheumatoid arthritis, psoriatic arthritis, gout, ankylosing spondylitis, osteoporosis, osteopenia and associated fragility fractures, traumatic fractures and sarcopenia. In particular the musculoskeletal disorder is osteoarthritis, more particularly osteoarthritis of the knee.

In some embodiments the compositions including the galectin inhibitor can inhibit any of the presently known 12 galectins, such as but limited to galectin-1, galectin-2, galectin-3, galectin-4, galectin-7, galectin-8, galectin-9, and galectin-12, and particularly galectin-3. The galectin inhibitor can be an antibody, peptide, antagonists/glycomimetic antagonists, small molecule, or functional derivative thereof. In some embodiment the inhibitors are antisense nucleic acids that target expression of one or more galectins, including miRNA, siRNA, antisense-RNA, small non-coding RNA. In another embodiment, a galectin inhibitor for use in the current compositions and methods or they can be aminopyrimidine-galactose hybrids. Exemplary commercially available inhibitors include, but are not limited to, GB1107, OTX008, 3-C12 (TFA), G3-C12, Thiodigalactoside, and TD139 or derivatives thereof.

Galactoarabino-rhamnogalacturonan (GR) and galactomannan (GM) were found to be potent inhibitors of galectin-3 with proven anti-inflammatory and anti-fibrotic properties in clinical studies [8]. However, additional data suggests that galectin-3 has the opposite effect. Galectin-3 is also a specific, high-affinity binding partner for lubricin. Considering the known ability of galectin-3 to crosslink glycoproteins, it is possible that galectins could augment joint lubrication via biomechanical stabilization of the lubricin boundary layer. It was found that competitive inhibition of galectin binding results in lubricin loss from the cartilage surface, and addition of multimeric galectin-3 enhances cartilage lubrication. In addition, galectin-3 showed low affinity for the surface layer of osteoarthritic cartilage and reduced affinity for sialylated O-glycans, a glycophenotype associated with inflammatory conditions. Taken together, this data suggests that galectin-3 reinforces the lubricin boundary layer; which, in turn, enhances cartilage lubrication and may delay the onset and progression of arthritis [20].

Described herein is the use of hyaluronic acid (HA) in combination with galectin inhibitors in compositions and methods of use thereof to treat or reduce musculoskeletal disorders. HA is a naturally occurring polysaccharide present in the human body. Due to its viscosity, elasticity and other rheological properties, HA is seen to exhibit lubricating and cushioning properties, which makes it a good candidate for use in various joint disorders.

In particular embodiments of the described compositions and methods, chitosan is used in combination with HA and/or galectin inhibitors. Chitosan is a naturally occurring polysaccharide, fibrous substance found mainly in the exoskeletons of crustaceans, and is produced by deacetylation of chitin. Chitosan has been used to treat or associated with the ability to decrease bleeding, weight loss, Crohn's disease, periodontitis, lower cholesterol and other implications. The described combinations of CM, HA and galectin inhibitors can be used in the above conditions as well.

In some embodiments, the described compositions include anti-inflammatory agents. Examples of anti-inflammatory agents are, but not limited to, NSAIDs, anti-leukotrienes, ImSAIDs, cytokines, and others, Together, compositions including the described anti-inflammatory agents can be used in treatment of musculoskeletal disorders.

In particular embodiments the described combination includes anti-pain compounds, including but not limited to opiates, acetaminophen and the like, and aspirin and the like. In some embodiments the opiate is naturally occurring, semi-synthetic, or synthetic. In particular non-limiting embodiments the opiate is heroin, fentanyl, morphine, codeine, hydrocodone, methadone, oxycodone, oxymorphone, tapentadol and others.

In some embodiments the described treatment includes the use of steroids. In specific non-limiting embodiments, the steroid is corticosteroids, anabolic-androgenic, or gonadal steroid, which can be administered in sequence with the described combinations or concurrently.

In particular embodiments the described treatment includes anti-fibrotic agents. In particular non-limiting embodiments the anti-fibrotic agent is galactoarabino-rhamnogalacturonan (GR), galactomannan (GM), Nintedanib, Pirfenidone, relaxin, Hydronidone, or derivatives thereof.

In particular embodiments the described components of the treatment are mixed together by simple laboratory mixing procedures. In some embodiments the components are joined by a linker. In some embodiments the CM is linked to a galectin inhibitor, such as but not limited to GM or GR, or a derivative thereof.

In particular embodiments the described treatment further includes administration of mesenchymal stem cells, adipose derived stromal cells, chondrocytes, platelet-rich plasma or whole blood. Such administration can occur in particular embodiments, concurrently, before, or after administration of the galectin-inhibiting composition.

In yet another embodiment, the compositions can be formulated in a sustained release formulation or system. In such formulations, the treatment is administered for an extended duration of time, such as 1, 2, 3, 4 or more days, including 1-72 hours, 24-48 hours, 16-36 hours, 12-24 hours, and any length of time in between. In particular embodiments, sustained release formulations are immediately available upon administration, and provide an effective dosage of the therapeutic composition, and remain available at an effective dosage over an extended period of time. In other embodiments, the sustained release formulation is not immediately available within the subject and only becomes available, providing a therapeutically effective amount of the active compound(s), after the formulation is metabolized or degraded so as to release the active compound(s) into the surrounding environment.

The therapeutic compounds and compositions of the present disclosure can be administered at about the same dose throughout a treatment period, in an escalating dose regimen, or in a loading-dose regime (e.g., in which the loading dose is about two to five times the maintenance dose). In some embodiments, the dose is varied during the course of a treatment based on the condition of the subject being treated, the severity of the disease or condition, the apparent response to the therapy, and/or other factors as judged by one of ordinary skill in the art. In some embodiments long-term treatment with the drug is contemplated.

The amount of the compound for use in the described treatment for musculoskeletal disorders, and that will be effective, will depend on the nature of the disorder or condition to be treated, as well as the stage of the disorder or condition. Therapeutically effective amounts can be determined by standard clinical techniques. The precise dose to be employed in the formulation will also depend on the route of administration, and should be decided according to the judgment of the health care practitioner and each patient's circumstances. The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the host undergoing therapy.

In particular embodiments, the described compositions can be formulated for immediate release, whereby they are immediately accessible to the surrounding environment, thereby providing an effective amount of the treatment, upon administration to a subject, and until the administered dose is metabolized by the subject.

Various delivery systems are known and can be used to administer the described compounds for use in the described treatment for musculoskeletal disorders or as part of the described methods. Methods of administration of the compounds include, but are not limited to, intrathecal, intradermal, intramuscular, intraperitoneal (ip), intravenous (iv), subcutaneous, epidural, parentally and oral routes. The therapeutic compositions can be formulated for administration by any convenient route, and depending on the target for therapeutic effect including, for example, infusion or bolus injection, and topical administration. Direct injection of the described compounds into the afflicted site is also contemplated herein.

In a specific embodiment, it may be desirable to administer the described pharmaceutical treatments by injection, catheter, suppository, or implant (e g , implants formed from porous, non-porous, or gelatinous materials, including membranes, such as sialastic membranes or fibers), and the like. In another embodiment, therapeutic agents are delivered in a vesicle, in particular liposomes for certain described galectin inhibitors, for example.

In particular embodiments, the compositions described herein can be supplied in any pharmaceutically acceptable composition. In such embodiments, the described components, such as CM, HA, and a galectin inhibitor, either alone or combined, are provided in a pharmaceutical formulation having a therapeutically effective dose of each therapeutic agent, as described herein, and including standard pharmaceutically acceptable salts, excipients, fillers and the like.

In one embodiment, a pump may be used. In another embodiment, the sustained released formulations include polymeric materials commonly used in the art, such as in implants, gels, capsules, and the like. By way of example, polymers such as bis(p-carboxyphenoxy)propane-sebacic-acid or lecithin suspensions may be used to provide sustained localized release. Other sustained release formulations and systems for use with the described components include those such as discussed in the review by Langer (Science 249, 1527 1990).

According to an embodiment of the present invention, the treatment is can be presented in unit dosage form and prepared by any of the methods well known in the art of pharmacy. In an embodiment of the invention, the unit dosage form is in the form of an ampoule, vial, or pre-filled syringe.

In particular embodiments the described combination of CM, HA and/or a galectin inhibitor can be engineered into a biodegradable scaffold, for example to be implanted in an afflicted knee, or the site of the damaged disc. Such biodegradable scaffolds are common in the art and are described in U.S. Pat. No. 8,469,968B2, U.S. Pat. No. 8,753,391B2, JP2018520761A, U.S. Pat. No. 8,287,594B2 and the like. Such scaffolds are formulated for sustained release of the described compounds over the course of days, or months.

In particular embodiments, the described compositions are formulated in pharmaceutically acceptable compositions of the compounds, using methods well known to those with skill in the art. For instance, in some embodiments, the compounds are formulated with a pharmaceutically acceptable carrier. The term “pharmaceutically acceptable” means approved by a regulatory agency of the federal or a state government or listed in the U.S. Pharmacopoeia or other generally recognized pharmacopoeia world-wide for use in animals, and, more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, hemp oil and the like. Saline solutions, blood plasma medium, aqueous dextrose, and glycerol solutions can also be employed as liquid carriers, particularly for injectable solutions. The medium may also contain conventional pharmaceutical adjunct materials such as, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like.

Examples of pharmaceutical excipients include starch, glucose, lactose, maltitol, sucrose, gelatin, malt, rice, flour, chalk, silica gel, sodium stearate, glycerol monostearate, talc, sodium chloride, dried skim milk, glycerol, propylene, glycol, water, ethanol, and the like. The described compositions can, if desired, also contain minor amounts of wetting or emulsifying agents, or pH buffering agents. The described compositions can take the form of solutions, suspensions, emulsion, tablets, pills, capsules, powders, all in immediate and sustained-release formulations as understood in the art. The therapeutic can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like.

Therapeutic preparations will contain a therapeutically effective amount of at least one active ingredient, preferably in purified form, together with a suitable amount of carrier so as to provide proper administration to the patient. The formulation should suit the mode of administration.

The ingredients of the described formulations can be supplied either separately or mixed together in unit dosage form, for example, in solid, semi-solid and liquid dosage forms such as tablets, pills, powders, liquid solutions, or suspensions, or as a dry lyophilized powder or water free concentrate in a hermetically sealed container such as an ampoule or sachet indicating the quantity of active agent. Kits comprising the described galectin inhibitors, CM and HA are accordingly also contemplated herein.

In particular embodiments the combination therapies for use in the described treatments and in the described methods of treatment include, but are not limited to combinations of GM and/or GR with HA and chitosan and other anti-inflammatory agent(s), or another galectin-3 inhibitor, with or without HA and/or Chitosan.

The following examples are provided to illustrate certain particular features and/or embodiments. These examples should not be construed to limit the disclosure to the particular features or embodiments described.

### EXAMPLES

Musculoskeletal disorders, and particularly osteoarthritis, are multi-factorial diseases that affect widely a population of different ages, with difference in disease stages, and with different etiology, a combinational approach that utilizes several therapeutic methods addressing different degrees of severity is required and is shown to be effective herein.

The present disclosure utilizes the ability of anti-inflammatory and anti-fibrotic agents, such as GM or GR with or without chitosan and/or with or without HA for a next-generation more effective treatment for osteoarthritis. Disclosed herein demonstrate the use of GM and GR, with or without HA and chitosan result in an enhanced healing and restoration of function of the OA knee compared to the standard treatment in the field, HA treatment.

### Example 1

### Reduction of Pain and Recovery of Weight Bearing Capacity in Knee Osteoarthritis (OA) by Treatment with HA−CM−GM

An animal pain model was used to surgically induce osteoarthritis (OA) in wistar rats. Knee OA was inflicted by partial medial meniscectomy on the right leg of 12 wistar rats while the contralateral knee served as self-control. Study design included 2 groups, 6 animals per group and 13 days of data was collected over the period of 6 weeks. After the procedure the animals were divided to the following groups: 1) HA treated and 2) HA−CM−GM treated. N=12. The positive control group, HA group, was treated at day 0 and day 14 post-surgery. The HA−CM−GM was treated only once at day 14 post procedure. Longitudinal data of relative weight bearing was calculated and normalized to weight bearing at day 14, the day of initiation of treatment with HA−CM−GM. Dosing was performed by injection to the OA knee, with an injection volume of 150 microliters.

Formulation for dosing of control group was taken from commercially available HA-1% viscosupplementation injection. CM−HA was prepared by mixing, in accordance with Patchor's standard procedures as described in International Patent Publication No.—WO 2009/150651 A1. CM−HA−GM was prepared immediately prior to usage by mixing CM−HA with: Davanat®(GM-CT-01 Lot #7277-D10-4034-0702 60 mg/mL, provided by Galectin Therapeutics) at ratio of CM:HA:GM-0.5:0.2:2.0.

The experimental outcome was assessed by measuring weight bearing on the OA inflicted right leg (RL) using an incapacitance tester, as well as the animal body weight. Regarding the incapacitance tester, the ratio of weight distribution between operated and non-operated paw is a natural measurement of the level of discomfort in the operated paw of a rodent and allows the objective measurement of spontaneous pain by assessing the postural equilibrium.

Measurements of the weight bearing of each animal in the incapacitance tester were generally taken in triplicates twice a week and their average was used for data analysis.

As shown in FIG. 1, the results of the present example demonstrate the statistically significant, surprising, and superior ability of treatment with CM−HA−GM, versus treatment of HA alone, to reduce pain detected by higher weight bearing capacity in the treated legs (p=0.005). The CM−HA−GM showed full restoration of the weight bearing capacity compared to that of prior to the surgically induced OA, indicating the regenerative property of CM−HA−GM, and potential therapeutic usage of CM−HA−GM.

### Example 2

### Attenuation of Irritation and/or Inflammation in Muscle Tissue Following Implantation of CM−HA Combined with GM or GR

This example shows that the addition of an anti-inflammation and/or anti fibrotic agent, such as GM or GR can reduce the onset and degree of inflammatory response that may be induced by implantation of CM-based formulations into muscle.

An aseptically produced CM is mixed with HA at a ratio of 0.6:0.3 resulting in a liquid solution at pH 7.3, which is then cross-linked with 0.1% genipin and implanted into muscle tissue of 3 rabbits to evaluate the local tissue response. Total of 0.4 ml of the CM+HA+Genipin is injected into 4 sites. Animals are euthanized 2 weeks post implantation; muscle tissue is excised and examined. Macroscopical examination show normal tissue response, however, microscopical examination show excessive inflammation, evident by abundance of white blood cells.

To attenuate the inflammatory response, genipin is replaced in the CM−HA by an anti-inflammatory and anti-fibrotic agent GM, GR or a combination of both. A total of 0.4 ml is implanted intra-muscularly (IM) in each rabbit, and compared to CM+HA+Genipin.

The results of this experiment are consistent with other observations described herein, and show that 2 weeks post implantation, animals treated with CM−HA+GM and/or GR display attenuation on inflammatory response compared to CM+HA+Genipin.

### Example 3

### Treatment of Osteoarthritis by Injection of GR, and GR-HA Compared to HA

Induction of knee osteoarthritis was performed by an anterior cruciate ligament transaction (ACLT) with medial meniscectomy (MMx) on one leg of wistar rats while the contralateral knee served as self-control. After the procedure the animals were divided into three experimental groups of 7 animals per group. Control group was treated by injection of 150 microliters of 1% HA into the knee. Experimental groups were treated by injection of 150 microliters of either GR or GR-HA.

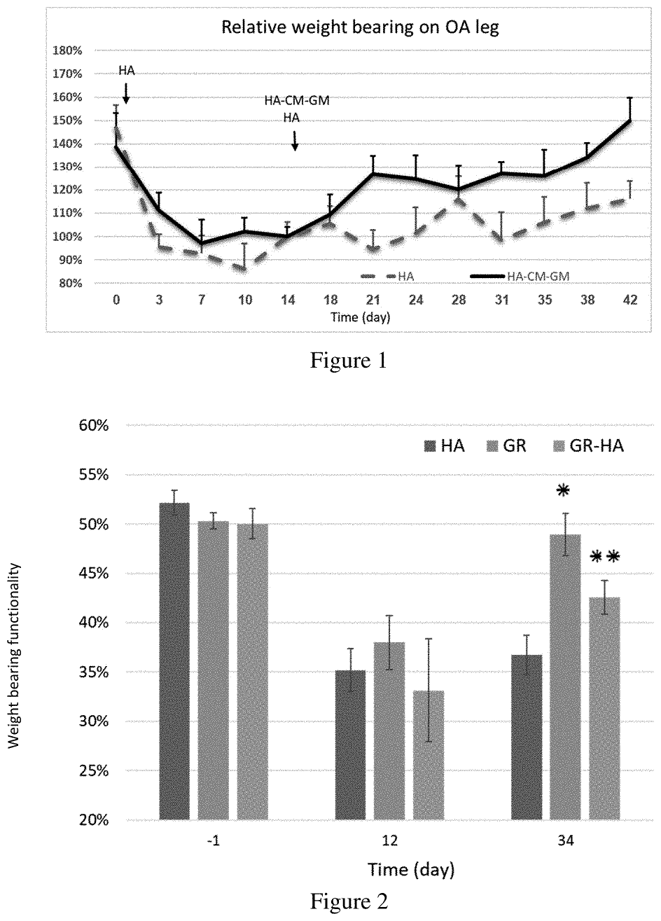

Formulation for dosing of control group was taken from commercial HA-1% viscosupplementation injection (Arthrease 1% sodium hyaluronate Lot R10008G, produced by Bio-Technology General). GR+HA was prepared by Patchor's standard procedures, as described in International Patent Publication No. WO 2009/150651 A1, of mixing HA with GM or with GR. All injections volume was 150 microliters. The positive control HA group was treated immediately post surgery and again at day 14 post surgery, and the HA−CM−GM was treated only once at day 14 post surgery. The experimental outcome was assessed by measuring weight bearing on the OA leg using an incapacitance tester. As shown in FIG. 2, data was collected before the operation (1 day pre-surgery, day-1), before the treatment (day 12) and 20 days post treatment (day 34). Results show significant increase in weight bearing ability in GR and GR-HA groups compared to HA group (*p=0.0012, **p=0.0458).

The results obtained demonstrate the superiority of the GR, and GR+HA groups compared to HA alone in restoration of weight bearing functionality of the operated leg. This experiment shows the potential therapeutic applicability of treating osteoarthritis, and other musculoskeletal diseases.

### Example 4

### GR Treated Animals Show Reduced Nociceptive Threshold

As described in Example 3, OA was induced on one leg of wistar rats while the contralateral knee served as self-control. Thereafter, 14 rats were divided into two experimental groups. 7 were treated by injection of HA to the knee, as a positive control, and 7 were injected with GR. Injection volume was 150 microliters.

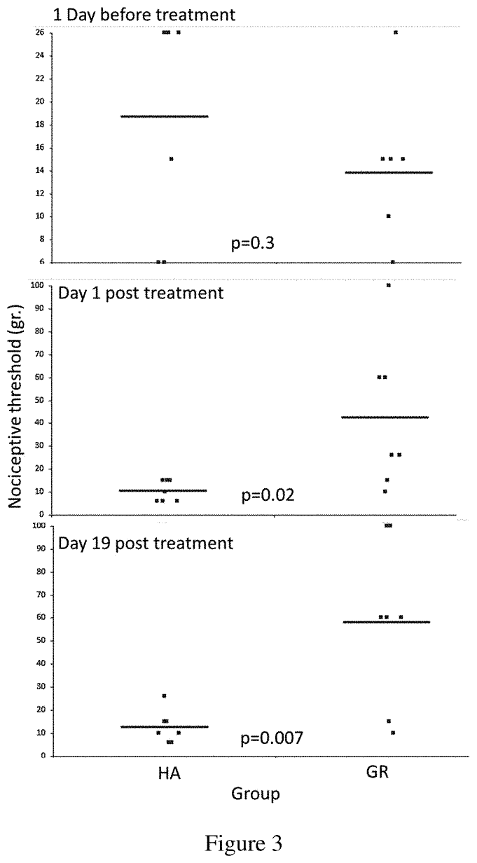

The experimental outcome was assessed using the Von Frey filament method for detection of mechanical nociceptive threshold test. Results show significantly reduced pain response of the GR group compared to control HA. As shown in FIG. 3, a day before treatment OA legs in both groups showed non-statistically significant difference in pain reaction (p=0.3). At 1 day and at 19 days post treatment there was a significant reduction in nociceptive threshold in GR treated group with statistically significant difference from the control HA (p=0.02, and p=0.007, respectively). No difference in nociceptive reaction was detected in the healthy legs of both groups (data not shown).

The results obtained demonstrate the superiority of treatment with GR compared to the standard treatment, HA, to reduce the nociceptive threshold. This experiment shows the potential therapeutic applicability of treating osteoarthritis, and other musculoskeletal diseases.

### REFERENCES

1—Arthritis: The Nation's Most Common Cause of Disability Jan. 30, 2010 at the Wayback Machine. The CDC.
2—Issue Brief Prepared by the Arthritis Foundation Spring 2016, file:///D:/AOM/Patchor/Grants/Arthritis-Military-Department-of-Defense-Funding-Issue-Brief.pdf].
3—Adae O Amoako et al, 2014 May 22. Clin Med Insights Arthritis Musculoskelet Disord. 2014; 7: 27-32
4—http://www.smith-nephew.com/key-products/sports-medicine/bst-cargel/
5—Ghazi Zadeh et al. 2017, Regen Med Ther 2017,1(1):16-29.
6—Front Mol Neurosci. 2018; 11: 158. Jian Jing Siew et al.
7—Yong Hu et al. 2017, Joint Bone Spine, Volume 84, Issue 1, January 2017, Pages 15-20
8—PLoS One. 2013 October 9;8(10):e75361.2013. Traber PG, et al.
9—Pomonis, J. D. et al. Pain. 2005; 114: 339-346
10—Ann N Y Acad Sci. 2010 January;1183:158-82. Liu FT1, Rabinovich GA.
11—Andersson G B. Lancet. 1999;354:581-585.
12—Cheung K M, et al. Spine. 2009;34:934-940
13—Brinjikji W, et al. AJNR Am J Neuroradiol. 2015;36:811-816.
14—Borenstein D G, et al. SW. http://www.ncbi.nlm.nih.gov/pubmed/11568190. J Bone Joint Surg Am. 2001;83:1306-1311
15—Long term outcome and adjacent disc degeneration after anterior cervical discectomy and fusion with titanium cylindrical cages. Sugawara T, et al. Acta Neurochir (Wien) 2009;151:303-309.
16—Maldonado C V, et al. Eur Spine J. 2011;20:403-407
17—Kelly M P, et al. Spine. 2011;36:1171-1179.
18—Predictive Factors of Successful Percutaneous Cervical Nucleoplasty for the Treatment of Pain with Cervical Herniated Disk, Min Kyoung Kim, et al.
19—Biological Treatment Approaches for Degenerative Disc Disease: A Review of Clinical Trials and Future Directions, Brenton Pennicooke, et al.
20—Galectin-3 Binds to Lubricin and Reinforces the Lubricating Boundary Layer of Articular Cartilage, Heidi L. et al.
21—Phys Sportsmed. 2016;44(2):101-108. Richards M M, et al.
22—Mediators Inflamm. 2018;2018:9186940. 2018 May 21. Brinchmann M F, et al.
23—Adv Orthop. 2012;2012:979152. Patchornik S, et al.
24—ATEQUAL 2010. 10.1109/ATEQUAL.2010.31. Robinson, Dror et al.(2010).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for treatment of a musculoskeletal disorder comprising:
administering to a subject in need thereof a composition comprising a therapeutically effective amount of at least one galectin inhibitor, thereby treating the disorder.

2. The method of claim 1, wherein the galectin inhibitor is selected from galactoarabino-rhamnogalaturonan (GR) and derivatives thereof, and galactomannan (GM) and derivatives thereof.

3. The method of claim 1, wherein the composition further comprises hyaluronic acid (HA).

4. The method of claim 1, wherein the composition further comprises chitosan up to 1000 kD, a chitosan mixture (CM), or chitosan oligomers of 1-50 kD or a derivative thereof.

5. The method of claim 2, wherein the composition further comprises chitosan up to 1000 kD, a chitosan mixture (CM), or chitosan oligomers of 1-50 kD or a derivative thereof.

6. The method of claim 1, further comprising administering to the subject at least one anti-inflammatory agent, pain-reducing agent, steroid, and/or anti-fibrotic agent.

7. The method of claim 1, wherein the galectin inhibitor inhibits at least one galectin selected from the group consisting of galectin-1, galectin-2, galectin-3, galectin-4, galectin-7, galectin-8, galectin-9, and galectin-12.

8. The method of claim 1, wherein the composition is formulated for oral administration, parental administration, transdermal administration or by direct injection.

9. The method of claim 1, wherein the composition is formulated for sustained release or extended release.

10. The method of claim 1, wherein the musculoskeletal disorder is selected from the group consisting of arthritis, osteoarthritis, rotator cuff tear, degenerative disc disease, and osteoarthritis of the knee.

11. The method of claim 1, wherein the subject is a human or non-human subject.

12. The method of claim 5, wherein the composition comprises chitosan mixture (CM), HA and GM provided at a ratio of about 0.4:0.2:2.0.

13. The method of claim 5, wherein the composition comprises CM and HA provided at a ratio of about 0.6:0.3, GM, and optionally GR.

14. The method of claim 1, further comprising administering to the subject mesenchymal stem cells, adipose derived stromal cells, chondrocytes, platelet-rich plasma or whole blood.

15. The method of claim 1, wherein the composition is implanted into the subject as a biodegradable scaffold.

16. A composition comprising CM, HA, and GM at a ratio of about 0.4: 0.2: 2.0.

17. A composition, comprising:
CM and HA at a ratio of about 0.6:0.3, and
GM and/or GR.
